(19) United States
(12) Patent Application Publication
(10) Pub. No.: US 2020/0046446 A1
(43) Pub. Date: Feb. 13, 2020

(54) DEVICES AND METHODS FOR NEEDLE DISPOSAL

(71) Applicant: BLEXX TECHNOLOGY, LLC, RHINELANDER, WI (US)

(72) Inventor: Leo Ronald KROMHOLZ, HARSHAW, WI (US)

(21) Appl. No.: 16/604,273

(22) PCT Filed: Apr. 13, 2018
(86) PCT No.: PCT/US2018/027576

§ 371 Date: Oct. 10, 2019

Related U.S. Application Data
(60) Provisional application No. 62/485,032, filed on Apr. 13, 2017.

(51) Int. Cl. A61B 50/36; A61M 5/32; B02C 19/00

(52) U.S. Cl. CPC A61B 50/362; A61M 5/3278; A61M 2005/3282; A61B 2050/364; B02C 19/0075

(57) ABSTRACT

Provided herein are devices, systems, and methods for disposal of needles. In particular, provided herein are devices, systems, and methods for safe and efficient disposal of medical and research needles.

The present application claims priority to U.S. Provisional Patent Application Ser. No. 62/485,032, filed Apr. 13, 2017, the disclosure of which is herein incorporated by reference in its entirety.

### FIELD

Provided herein are devices, systems, and methods for disposal of needles. In particular, provided herein are devices, systems, and methods for safe and efficient disposal of medical and research needles.

### BACKGROUND

Health care workers, lab workers, sanitation workings, and housekeeping staff are at substantial risk of acquiring bloodborne pathogen infections through exposure to blood or infectious body fluids. Common pathogens obtained from needle sticks include hepatitis B virus, Hepatitis C virus, and human immunodeficiency virus.

One recent study found that 35.6% of Egyptian health workers were exposed to at least 1 needlestick injury during the past 3 months with an estimated annual number of 4.9 needlesticks per worker (Talaat et al, AJIC, 31: 469). Further studies revealed high levels of needle sticks and unsafe handling of needles among health care workers in India (Muralidhar et al., Indian J Med Res 131, March 2010, pp 405-410), the UK (Thomas and Murray, Ann R Coll Surg Engl 2009; 91: 12-17), and Malaysia (Norsayani and Hassim, Ann R Coll Surg Engl 2009; 91: 12-17). In addition, sanitation workers and housekeeping workers are at risk during disposal of needles in household and other trash collection (e.g., from home or recreational users of needles).

Many needlestick injuries occur during disposal of needles by users that do not follow appropriate disposal protocols (e.g., re-capping needles, failing to use a proper disposal container, or misuse of disposal containers). While this problem has been prevalent for many, many, years, it remains unsolved. Improved disposal systems and methods are needed in order to reduce injuries and infection.

### SUMMARY

Provided herein are devices, systems, and methods for disposal of needles. In particular, provided herein are devices, systems, and methods for safe and efficient disposal of medical and research needles.

The devices and methods described herein solve the problem of safe and efficient needle disposal by providing a small, inexpensive, and convenient method of safely disposing of needles at the location of use. This eliminates risk of pathogen exposure for both medical workers and waste disposal workers.

For example, in some embodiments, the present disclosure provides a needle disposal device or system comprising: a) a grinder comprising a grinding surface, said grinder operably linked to a motor (e.g., via a shaft attached to the grinder) configured to spin the grinder; b) a housing enclosing said grinder; c) an access port in said housing configured to align a needle inserted into the access port with said grinding surface of said grinder; and d) a waste containment component. In some embodiments, the grinding surface is a disc or drum. In some embodiments, the grinding surface comprises a material selected from, for example, ceramic, metal, aluminum oxide particles, silicone carbide, or diamond. In some embodiments, the motor is powered by AC or DC. In some embodiments, the DC is provided by a disposable or rechargeable battery. In some embodiments, the access port further comprises one or more air tight seals. In some embodiments, the seal is composed of rubber or silicon. In some embodiments, the seal comprises one or more access holes configured to allow a needle and a needle attachment portion of a syringe to enter the hole. In some embodiments, the hole does not allow the barrel of the syringe to enter the hole. In some embodiments, the waste containment component comprises at least one magnet. In some embodiments, the at least one magnet is a permanent or temporary magnet. In some embodiments, the waste containment component further comprises a removable cap (e.g., comprising an air tight seal). In some embodiments, the at least one magnet is disposable or re-usable. In some embodiments, the housing further comprises a UV light configured to irradiate a needle or waste product generated by the grinder destroying a needle. In some embodiments, the housing is composed of metal or plastic. In some embodiments, the device is 1 to 12 inches in length, 1 to 6 inches in width, and 1 to 6 inches in height (all values therein between are contemplated).

Further embodiments provide a method of disposing a needle, comprising: a) inserting the needle into the needle port of the device described herein; and b) activating the grinder of the device such that the needle is destroyed and metal waste from the needle is collected in the waste containment component (e.g., attracted by a magnet in the waste containment component). In some embodiments, the grinder is activated with a user controlled switch or automatically (e.g., with a sensor that detects needle entry into the access port or hole or in the proximity to the grinder). In some embodiments, the method further comprises the step of removing the magnet and disposing of the magnet or removing the metal waste from the magnet and returning the magnet to the device. In some embodiments, waste is removed via a vacuum in communication with said waste containment component. In some embodiments, waste is collected in a chamber, bag, etc. In some embodiments, the method further comprises the step of activating the UV light to inactivate any pathogens or other biohazards on the needle or needle fragments or debris. In some embodiments, the device grinds the needle until all of the needle is removed from a syringe attached to the needle. In some embodiments, the needle is converted to particulate debris (e.g., dust) and or fragments. In some embodiments, the device is used in a home, a hospital, a nursing home, a public restroom, a research lab, a hotel, a clinic, or an ambulance.

Additional embodiments are described herein.

### BRIEF DESCRIPTION OF THE DRAWINGS

FIG. 1 shows a side view of an exemplary device of embodiments of the present disclosure.

FIG. 2 shows a top view of an exemplary device of embodiments of the present disclosure.

FIG. 3 shows a side view of an exemplary device comprising a vacuum component.

FIG. 4 shows an exemplary waste containment component of devices described herein comprising a magnet.

FIG. 5 shows an expanded view of a needle access port of devices of embodiments of the present disclosure.

### DETAILED DESCRIPTION

Provided herein are devices, systems, and methods for disposal of needles. In particular, provided herein are devices, systems, and methods for safe and efficient disposal of medical and research needles.

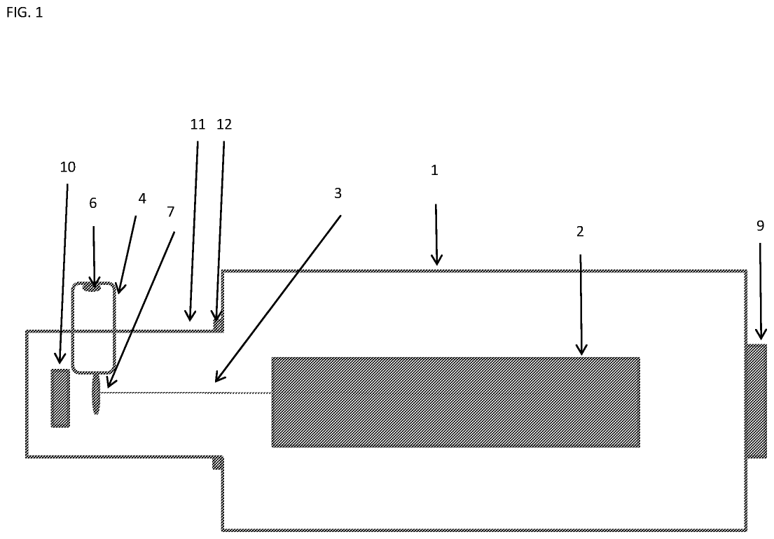

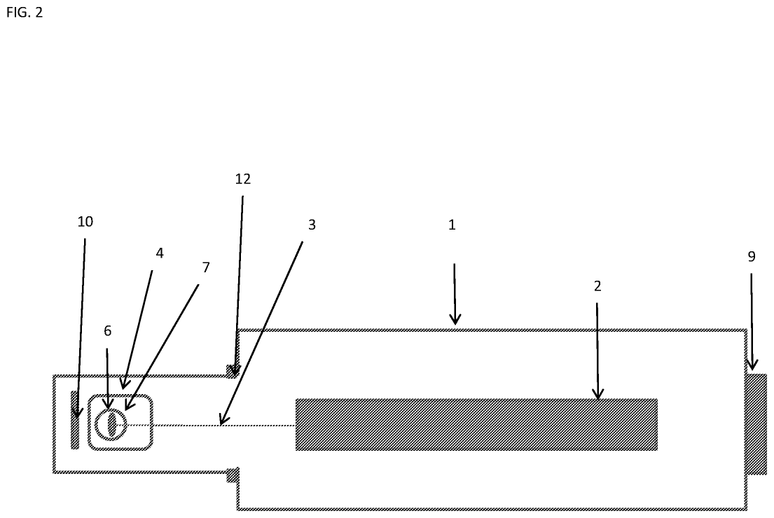

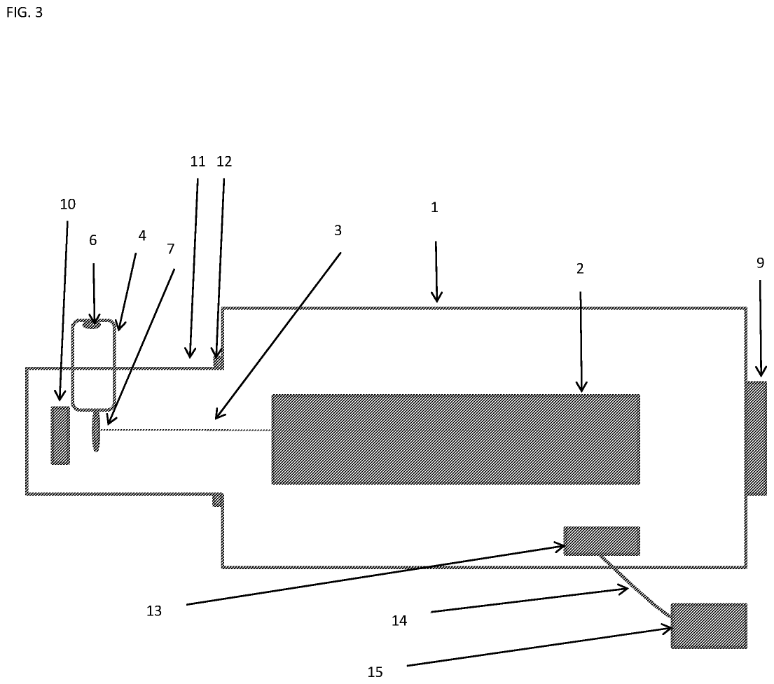

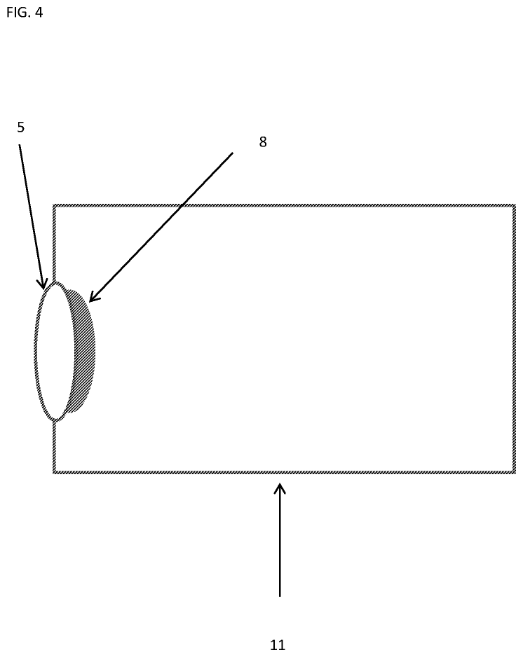

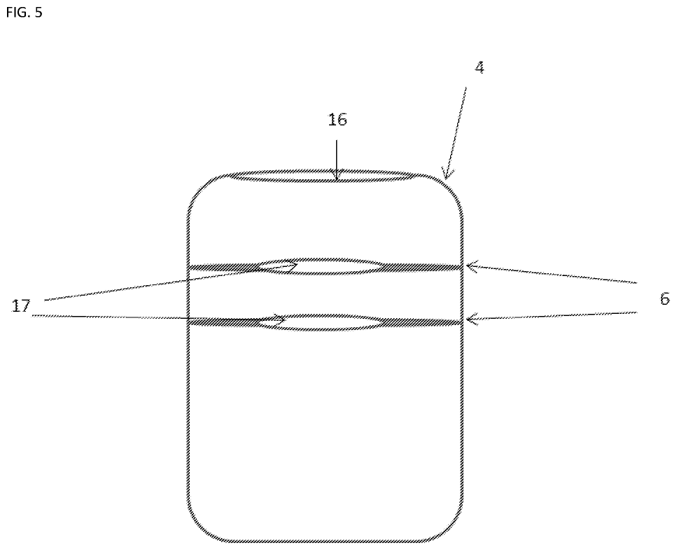

FIGS. 1 and 2 shows a side view (FIG. 1) and top view (FIG. 2) of exemplary devices. Device 1 comprises motor 2, power source 9, and needle grinder (e.g., disc or drum) 7 having a shaft 3 connected to the motor 2. Devices (See e.g., FIG. 5) further comprise needle access port 4 with opening 16 and optional seal (s) 6. In some embodiments, the seal comprises access hole 17 for insertion of needle and a portion of the syringe. In some embodiments, devices comprise a waste containment component 11. In some embodiments (See e.g., FIG. 4), the waste containment component comprises at least one (e.g., at least one, two, three, four, five, or more) magnet 8 affixed to removable cap 5. In some embodiments (See e.g., FIG. 3), the waste containment component comprises a vacuum 13 connected via tubing 14 to collection component 15. In some embodiments, the entire waste containment component is removable (e.g., via attachment component 12) and disposable.

The present disclosure is not limited to particular device components. In some embodiments, the power source is an AC or DC power source. In some embodiments, the AC power source is hard wired into a circuit or plugged into an outlet. In some embodiments, a DC power source comprises a disposable or rechargeable battery. In some embodiments, the motor is a commercially available AC or DC motor. In some embodiments, the motor operates on a power range of 10-500 V (e.g., 100-200 V). In some embodiments, the motor is a rotary motor that rotates the shaft and grinder at a constant or variable RMP. The present disclosure is not limited to particular RPM values or ranges of RPM values for such motors. In some embodiments, the RMP of the motor is 1000-50,000 RPM (e.g., 5000-35,000 or other value). In some embodiments, the grinder is ceramic, metal, sandpaper (e.g. aluminum oxide particles), silicone carbide, diamond, etc.

The needle port allows a user to contact the used needle with the grinding disc or drum (or other shaped grinding surface). In some embodiments, the needle access port comprises an air tight seal (e.g., silicone or rubber) to prevent needle debris and any infectious agents from leaving the device. In some embodiments, the seal comprises an access hole sized to allow the needle and at least a portion of the syringe or spacer that attaches the needle to the syringe to enter. In some embodiments, the hole is sized to exclude all or a portion of the syringe barrel or a user's finger from the hole. In some embodiments, holes are 1-10 mm (e.g., 1-8 mm, 2-6 mm, 1-5 mm, etc.) in diameter. In some embodiments, the opening or seal is positioned to allow the needle to make contact with the grinding disk or drum in a suitable location. In some embodiments, the access port is covered by a cap or size-restrictive barrier that prevents fingers or other undesired objects from entering the access port until the device is ready for its intended use. Addition of a protective cap also provides an additional barrier to ensure that destroyed needle dust or fragments do not exit the device.

In some embodiments, devices comprise a waste containment system. In some embodiments, the waste containment system comprises a magnet (e.g., one or a permanent or temporary magnets). In some embodiments, the magnet is attached or proximal to a removable cap. In some embodiments, the magnet is a disk or other shape. In some embodiments, the magnet is disposable. For example, in some embodiments, ground pieces of needle attach to the magnet after grinding. When the magnet is full, it is then removed via the cap and disposed of or cleaned and then replaced. In some embodiments, the waste containment system further comprises a UV light 10 for sterilization of metal pieces (e.g. to inactivate viral particles in the device or on the magnet). Other decontamination techniques may also be employed alone or in contaminations with UV light or each other, including, but not limited to, exposure to heat (e.g., via addition of a heating component to the device or exposing the waste containment system to an external heat source, e.g., autoclave system), exposure to anti-pathogenic materials such as biocides, antiseptics, or antibiotics (e.g., alcohols (e.g., ethanol, isopropanol), aldehydes, anilides, biguanides, bispenols, diamidines, halogen-containing agents, halophenols, heavy metal derivatives (e.g., silver compounds), peroxygens, phenols and cresols, quaternary ammonium compounds, ethylene oxide, formaldehyde, hydrogen peroxide, etc.) that may be coated on, added to, or contained in the waste containment system. In some embodiments, heating caused by friction from the contact of the needle with the grinder provides decontamination.

In some embodiments, the waste containment system is removable (e.g., detachable) and disposable. In some embodiments, waste is removed via a vacuum attached to the device and attached to disposable or reusable collection component (e.g., bag, etc.). In some embodiments, a waste containment removal service is provided with the device. In some embodiments, at designated times or after a certain level of use, a professional service removes the waste containment system and adds a new one. In some embodiments, the device is configured with a counter (e.g., a sensor with a counter) that quantifies the number of needles destroyed or the volume of waste material collected so that removal and disposal of the waste containment system is timely made. The device may include an indicator (e.g., LED light, alarm, etc.) that communicates a full or near-full status to local users or may include a communication component that sends a full or near-full status alert to remote entities (e.g., service providers) via a communication network.

In some embodiments, the device is configured for mounting on or near a garbage can or other waste disposal device. As such, for example, after a needle is destroyed, the remaining portions of the syringe or needle-containing device may be disposed of in the garbage can or other waste disposal device. In some such embodiments, the device of the present invention comprises a mounting clip that permits the device to mount over a top edge of a garbage can.

In some embodiments, the device is enclosed in a housing (e.g., metal or plastic housing). In some embodiments, devices are compact (e.g., 1-20 inches in each dimension). For example, in some embodiments, devices are 1 to 12 inches in length (e.g., 2-10 inches, 3-5 inches, etc.), 1 to 6 inches in width (e.g., 1-5 inches, 2-4 inches, etc.), and 1 to 6 inches in height (e.g., 1-5 inches, 2-4 inches, etc.) although smaller or larger values are specifically contemplated.

In use, a user uncaps a needle and uses the needle as indicated (e.g., to inject a human or non-human subject). The present disclosure is not limited to particular needle types. Examples include, but are not limited to, medical needles with attached syringe, IV needles, blood collection needles, suture needles, etc. In some embodiments, the needle comprises a plastic or glass spacer that attaches the needle to the syringe. In some embodiments, the needle is any gauge commonly used in research or medicine (e.g., 14, 15, 16, 17, 18, 19, 20, 21, 22, 23, 25, 25, 26, 27, 27, 29, or 30 gauge). The user then inserts the needle into the needle port of a device described herein. The user activates the device (e.g., with a switch or automatically (e.g., when the needle contacts the grinder via a pressure or electrical sensor)). The needle is ground by the grinding disk or drum and any metal pieces (e.g., dust or fragments) are trapped on the magnet, taken up by a vacuum, or otherwise sequestered. In some embodiments, at least a portion of the syringe or spacer attaching the syringe to the needle is ground (e.g., to remove all metal from the syringe). Such methods greatly reduce the risk of accidental sticks by users or sanitation workers disposing of medical or household waste.

The devices described herein find use in a variety of settings (e.g., including, but not limited to, a home, a hospital, a nursing home, a public restroom, a research lab, a hotel, a clinic, or an ambulance).

The foregoing description of illustrative embodiments of the disclosure has been presented for purposes of illustration and of description. It is not intended to be exhaustive or to limit the disclosure to the precise form disclosed, and modifications and variations are possible in light of the above teachings or may be acquired from practice of the disclosure. The embodiments were chosen and described in order to explain the principles of the disclosure and as practical applications of the disclosure to enable one skilled in the art to utilize the disclosure in various embodiments and with various modifications as suited to the particular use contemplated. It is intended that the scope of the disclosure be defined by the claims appended hereto and their equivalents.

1. A needle disposal device, comprising:
a) a grinder comprising a grinding surface, wherein said grinder is operably linked to a motor and shaft configured to turn said grinder;
b) a housing enclosing said grinder;
c) an access port configured to align a needle with said grinder; and
d) a waste containment component configured to retain needed debris.

2. The device of claim 1, wherein said grinder is a disc or drum.

3. The device of claim 1, wherein said grinder comprises a material selected from the group consisting of ceramic, metal, aluminum oxide particles, silicone carbide, and diamond.

4. The device of claim 1, wherein said motor is powered by AC or DC.

5. The device of claim 4, wherein said DC is provided by a disposable or rechargeable battery.

6. The device of claim 1, wherein said access port further comprises one or more air tight seals.

7. The device of claim 6, wherein said seal is rubber or silicon.

8. The device of claim 6 or 7, wherein said seal comprises one or more access holes.

9. The device of claim 1, wherein said waste containment component comprises at least one magnet.

10. The device of claim 9, wherein said at least one magnet is a permanent or temporary magnet.

11. The device of claim 9, wherein said at least one magnet is at least two magnets.

12. The device of claim 1, wherein said waste containment component further comprises a removable cap.

13. The device of claim 12, wherein said cap comprises an air tight seal.

14. The device of claim 1, wherein said at least one magnet is disposable or re-usable.

15. The device of claim 1, wherein said waste containment component is removable.

16. The device of claim 15, wherein said waste containment component is disposable.

17. The device of claim 1, wherein said device further comprises a vacuum.

18. The device of claim 1, wherein said waste containment component further comprises a UV light.

19. The device of claim 1, wherein said housing is metal or plastic.

20. (canceled)

21. A method of disposing a needle, comprising:
a) inserting said needle into the needle port of the device of claim 1; and
b) activating the grinder of said device such that said needle is destroyed and metal waste from said needle is collected is said waste containment component.

22-27. (canceled)
